(19) United States
(12) Patent Application Publication
(10) Pub. No.: US 2008/0132419 A1
(43) Pub. Date: Jun. 5, 2008

(54) DISSOLUTION AND PRECIPITATION OF COCRYSTALS WITH IONIZABLE COMPONENTS

(76) Inventor: Nair Rodriguez-Hornedo, Ann Arbor, MI (US)

Correspondence Address: HARNESS, DICKEY & PIERCE, P.L.C., P.O. BOX 828, BLOOMFIELD HILLS, MI, 48303, US

(21) Appl. No.: 11/867,432

(22) Filed: Oct. 4, 2007

Related U.S. Application Data
(60) Provisional application No. 60/849,330, filed on Oct. 4, 2006.

(51) Int. Cl. C40B 30/00; C07D 223/16

(52) U.S. Cl. 506/7; 540/589

(57) ABSTRACT

An approach to designing families of cocrystals with desired (tunable) pH dependent dissolution is developed. The solubility and dissolution rate of a family of cocrystals with the same API and a series of ligands that are weak acids or weak bases has been found to be determined and controlled by the acid or base dissociation constant of the ligand and the pH of the dissolution medium. In various aspects, pH dependent dissolution is imparted to a non-ionizable API or the dissolution of ionizable API's is modulated.

### CROSS-REFERENCE TO RELATED APPLICATIONS

This application claims the benefit of U.S. Provisional Patent Application No. 60/849,330 filed on Oct. 4, 2006. The disclosure of the above application is incorporated herein by reference.

### GOVERNMENT RIGHTS

The subject matter disclosed herein was developed with government support under Contract No. GM007767 awarded by the NIH. The Government has certain rights in the invention.

### FIELD OF THE INVENTION

This invention relates to reaction cocrystallization of molecular complexes or cocrystals. In particular, the invention relates to methods for preparing and screening cocrystals, wherein the cocrystal contains an ionizable component.

### BACKGROUND OF THE INVENTION

Cocrystallization is an essential processing step in the success of making multi-component crystalline phases (crystalline molecular complexes or cocrystals). Such multi-component crystalline phases have proved important in the pharmaceutical area, for example, where molecular complexes are known to form that contain an active pharmaceutical ingredient and that crystallize to give a unique crystal structure containing the molecular complex. Most known cocrystals contain two components, but three component cocrystals are known. Cocrystals include systems in which one or more of the formal components is itself made of more than one identifiable molecular form. These include cocrystal components that are salts, hydrates, solvates, and the like. As for stoichiometry, the most common types of cocrystals are 1:1 and 2:1 complexes, where the ratios indicate the stoichiometric presence of cocrystal components in the molecular complex or cocrystal.

The most generally applied techniques to prepare cocrystals are crystallization by solvent evaporation and by cooling solutions containing the individual components. Commonly used solution methods for cocrystal formation use organic solvents and solution concentrations of cocrystal components (reactants) on the same molar basis as the cocrystalline product. With these methods, there is a risk of crystallizing the single component phases thereby eliminating the possibility of accessing the multi-component crystalline phase. Moreover, crystallizing by the known solution methods entails the evaluation of a number of crystallizing conditions such as choice of solvent, component concentration, rate and extent of evaporation or cooling, and the like.

For compositions such as cocrystals that contain an active pharmaceutical ingredient (API), the pH dependence of dissolution is an important consideration. Depending on formulation and medical application, it is desirable under certain circumstances for an active ingredient to dissolve or be delivered in a pH region corresponding to that of the stomach (low pH about pH 1-3) or at higher pH regions such as in the duodenum or intestines (e.g. pH 6-8). Methods of tailoring or controlling the pH-dependent dissolution behavior of cocrystals or molecular complexes containing active pharmaceutical ingredients would be a significant advance.

### SUMMARY OF THE INVENTION

A method for preparing multi-component crystals (cocrystals) by reaction cocrystallization involves a chemical reaction or interaction of components of the cocrystal in a microphase or a macrophase and leads to formation of a crystalline product of multiple components, without the need for grinding, solvent evaporation, or temperature variation. As used herein, cocrystals refer to single phase compositions that are made of at least two components that are identifiable as different molecular forms. It does not refer to multiple crystals, i.e. crystals in which the crystallizing form is a dimer, trimer, or higher multimer of a single component.

Conditions are achieved leading to rapid cocrystallization (i.e. formation of a solid phase containing the cocrystal by precipitation) by the choice of reactant concentration in solutions, solvent, pH, and other factors. In one aspect, reaction cocrystallization is a term given to the process by which the apparent solubility of a multi-component complex in a solvent system is decreased upon adding molar excesses of one (but not both or all) of the components of the complex. In another aspect, pH is used to control cocrystal dissolution and/or precipitation when at least one cocrystal component is ionizable, either as a weak acid or a weak base. It is believed the methods work in part by reducing the solubility of the molecular complex in the solvent, increasing the likelihood that the molecular complex is the least soluble form in the system, upon which it precipitates.

In various aspects, the concept of a cocrystal solubility product is advanced to explain the phase solubility diagram of the cocrystal system and identify conditions under which cocrystals can be prepared in micro- and macrophases, or alternatively identify conditions under which formation or precipitation of cocrystals is desirably avoided. The effect of pH is also described.

In various embodiments, methods of producing cocrystals by precipitation from a liquid phase involve combining two or more reactants (cocrystal components) together with solvent in a molar ratio such that the molar concentration of one of the reactants in solution is significantly higher than the concentration of the other reactant or reactants. In preferred embodiments, the molar excess of one reactant over the other is greater than 2:1, and is preferably at least 5:1. As noted, it is believed that the molar excess of one of the reactants reduces the solubility of the complex by a mechanism analogous to the common ion effect.

In other embodiments, precipitation and/or dissolution of cocrystals is controlled as a function of pH. When one of the cocrystal components is an ionizable weak acid, the rate of dissolution is increased by increasing the pH of the dissolution media, and precipitation is enhanced (solubility is lowered) when the pH of a solution containing the components is lowered. When one of the components is a weak base, the rate of dissolution is increased by lowering the pH, and precipitation is enhanced (solubility of the cocrystal is lowered) when the pH of a solution containing the components is raised. In various embodiments, methods of large scale manufacture, precipitation, or harvesting of cocrystals is provided, without temperature change or solvent evaporation, by adjusting the pH of a solvent system containing the components of a cocrystalline complex.

Further areas of applicability of the present invention will become apparent from the detailed description provided hereinafter. It should be understood that the detailed description and specific examples, while indicating various embodiments of the invention, are intended for purposes of illustration only and are not intended to limit the scope of the invention.

### BRIEF DESCRIPTION OF THE DRAWINGS

FIG. 1 shows pH-solubility dependence for a cocrystal of a non-ionizable API and weakly acidic ligands with pKa values of 2 and 5. Cocrystal Kₛₚ=1×10⁻⁴ M².

FIG. 2 is a graph showing cocrystal solubility as function of pH for cocrystals with either acidic or basic ligand and non-ionizable API.

FIG. 3 shows effect of pKa of cocrystal components on cocrystal solubility (pKa1 for weak acid and pKa2 for conjugate acid of the weak base). There is a minimum solubility at pH values depending on the pKa of the acid and base components.

### DETAILED DESCRIPTION

It is known that under various conditions some organic compounds form nonionic complexes in solution with other organic compounds. The complexes are held together with nonionic interactions such as hydrogen bonding and the like. Under certain crystallization conditions, including those in which the complex thus formed is less soluble than the other forms in the system, the molecular complex crystallizes or precipitates out of a solution to form what is referred to as a cocrystal. As used here unless the context requires otherwise, the term cocrystal refers to the precipitated solid, while the more general term molecular complex is used for the multi-component molecular complex (usually a binary or ternary complex, with binary complexes being the most common) whether in the solution or solid phase. The molecular entities that interact with one another to form the molecular complexes and the cocrystals are referred to as cocrystal reactants, prospective reactants, cocrystal components, or similar terms. Although individual reactants can be provided in salt form, it is understood that the molecular complexes are formed by nonionic interactions between and among reactants, and do not rely on ionic interactions such as salt formation to form the complexes.

Solvents include single liquids and solvent systems containing two or more individual liquids where the liquids are aqueous or organic and act as solvent for the reactants or complexes. When separate solutions of individual reactants or components are provided, it is understood that the individual components can be in the same or in different solvent systems with respect to each other. It is further understood that while a solution of a particular reactant predominantly contains the reactant mentioned, it may also further comprise other components or minor amounts of other reactants in the system. The pH of aqueous solutions can be adjusted by additions of acids or bases or can be buffered as desired.

In some embodiments, reactants or components are provided in the physical form of slurries or suspensions. These are to be understood as containing a solid phase in contact with a liquid phase; when the component is soluble in the liquid (i.e., when the liquid acts as a solvent for the component), the liquid phase generally contains at least some dissolved solids corresponding to the reactant being used. In some aspects of the invention, the liquid phase of the slurry or suspension is saturated with respect to the reactant species present as the solid in the suspension or dispersion.

Although several aspects of the invention are described herein in theoretical chemistry terms, it is to be understood that the invention is not to be limited to the theory put forth. Theoretical considerations are presented in order to more fully describe the invention and its various uses.

The identity of the reactants or components making up the molecular complexes and cocrystals described herein is not particularly limited. However, the structure of the individual components must allow for some kind of nonionic interaction such as hydrogen bonding, dipole-dipole interactions, and the like to stabilize the complex. In various preferred embodiments of the invention, at least one of the reactants is an active pharmaceutical ingredient such as a drug or other pharmaceutical active agent. A number of molecular complexes and cocrystals of active pharmaceutical ingredients are known. In various embodiments, the invention provides methods of making known cocrystals and methods for screening to find new cocrystal systems.

Cocrystals or molecular complexes contain two or more components that are held together in the complex by nonionic interactions such as discussed above. That is, in the complex the components are uncharged and are distinguished from salts. Some cocrystal components are made of molecular species that cannot be ionized at any pH. However, other possible cocrystal components are ionizable as a charged species at certain pHs. For example, weak acids exist in a negatively charged anionic form at pH levels that are about equal to its pKa or higher. Examples of such weak acids include carboxylic acids. Similarly, weak bases include those molecular species that exist in a positively charged or protonated form below a pH that is approximately equal to the pKa of the conjugate acid of the weak base. Examples of weak bases include organic amines. For convenience, the pKa of the base conjugate acid will be referred to as the pKa of the corresponding (weak) base.

Cocrystal components are neutral if they do not contain an ionizable group such as are present in the weak acids and the weak bases. Non-ionizable organic groups include hydroxyls, organic amides, esters, ethers, and so-on. Such functional groups do not ionize under normal conditions, either at high pH or at low pH. Weak acids, on the other hand include those that have a pKa from about 1-14, while weak bases include those whose pKa is about 1-14. A weak base can equivalently be thought of as one whose conjugate acid is a weak acid.

In one embodiment, methods are provided for controlling the solubility of a crystalline complex in an aqueous solvent system. The crystalline complex is made of two or more organic reactants that are held in complex by non-ionic interactions among or between them, where one of the reactants of the complex is a weak acid or a weak base. An aqueous solvent system is one that contains water or a mixture of water and other solvent, where other solvents are present at a level that does not interfere with meaningful pH measurements of the solvent system. The method for controlling solubility encompasses methods for enhancing solubility of the crystalline complex on the one hand (for example to enhance a dissolution rate) and for decreasing the solubility of the complex on the other (for example to precipitate the solid cocrystal complex as part of a method of industrial manufacture). Exemplary embodiments include forming the molecular complex in the solvent system or combining the reactant components of the crystalline complex or cocrystal in the solvent system. Simultaneously or subsequently, the pH of the solvent system is then adjusted to or maintained at a pH that provides the desired solubility, rate of dissolution, or rate of precipitation of the complex in/from the solvent system.

When one of the components of the molecular complex is a weak acid, the pH maintenance or adjustment is to or at a value of less than or equal to about the pKa of that weak acid component in order to enhance precipitation of the molecular complex or decrease solubility. For example, the pH is the pKa+2, the pKa+1 or below to lower the solubility and favor precipitation. In some embodiments, it is favorable to reduce the pH even to the pKa or a unit or two below the pKa to achieve desired solubility/precipitation behavior. A pH at or below the pKa of the weak acid may be needed for the more soluble crystalline complexes, depending on the concentration and the solvent system.

When one of the components of the molecular complex is a weak base, the pH is treated in an inverse way. That is, the pH is adjusted to or maintained at a value that is about equal to or greater than the pKa of the weak base component in order to enhance precipitation or reduce solubility, and at or to values near or below the pKa of the weak base to enhance solubility. Depending on the solubility of the complex and the desired behavior, the pH adjustment or maintenance is at the pKa minus two units or above, at the pKa minus one unit or above, at the pKa itself or above, or even at the pKa plus one unit or above.

In various embodiments, the pH is adjusted to a value that is one unit below the pKa of a weak acid cocrystal component or one unit above the pKa of a weak base cocrystal component. In other embodiments, the pH is adjusted to or maintained at a value that is 2 or more units less than the pKa of a weak acid or that is 2 or more units greater than the pKa of a weak base.

In various embodiments, the molecular complex preferably precipitates at the pH at or below the pKa of the weak acid component, or in the case of a weak base at or above the pKa of the weak base. Ionizable weak acids include those containing a carboxylic acid group, while ionizable weak bases include those that contain an organic amine group. If desired, the method for controlling the solubility by maintaining or adjusting pH is carried out along with methods that provide combining a plurality of streams that collectively provide at least a 5:1 of more excess of one of the reactants, relative to the amount of reactant in the molecular complex. Preferably, the molar excess is at least 10:1. Advantageously, one or more of the reactants of the molecular complex is an active pharmaceutical ingredient.

The pH dependence of the solubility of cocrystals having a weak acid or weak based component can be exploited in methods for producing commercial quantities of cocrystals, especially those in which one of the reactants is an active pharmaceutical ingredient. Thus in various embodiments, there is provided a method for manufacturing a cocrystal, comprising adding the component reactants of the cocrystal to a solvent at a first pH, then adjusting the pH to a value at which the solubility of the crystalline complex formed between or among the reactants is lowered sufficiently that the crystalline molecular complex (the cocrystal) precipitates from the solvent. The precipitate can then be collected and dried by conventional means.

Consistent with the description of solubility behavior herein, when the cocrystal reactants include a weak acid, the pH is lowered to precipitate the solid cocrystal from the solvent system. Depending on the actual solubility of the complex, it may be desirable to lower the pH at least to the pKa plus two, at least to the pKa plus or at least to the pKa. The pH of precipitation is thus the pKa plus two units or lower, the pKa plus one unit or lower, the pKa or lower, the pKa minus one unit or lower, or the pKa minus two units or lower.

When the cocrystal reactants include a weak base, the pH is raised to precipitate the solid cocrystal from the solvent system. Depending on the actual solubility of the complex, it may be desirable to raise the pH at least to the pKa minus two units, at least to the pKa plus one unit, or at least to the pKa. The pH of precipitation is thus the pKa minus two units or higher, the pKa minus one unit or higher, the pKa or higher, the pKa plus one unit or higher, or the pKa plus two units or higher.

Dissolution of solid cocrystals is carried out in a way opposite of precipitating described above. That is, dissolution depends on increasing the solubility of the cocrystal as a function of pH. To increase the solubility of a cocrystal and to thereby increase the rate at which it dissolves, the pH is maintained at or adjusted to a value that increases the solubility of the cocrystal. For a cocrystal one component of which is a weak acid, such a pH is at about the pKa of the weak acid component, or preferably at least one or at least two units higher. For the case where the cocrystal has an ionizable component which is a weak base, such a pH is at about the pKa or preferably at least one or at least two units lower than the pKa of the weak base.

In one aspect, the current teachings provide an approach to the designing families of cocrystals having desired pH dependent dissolution properties. For example, cocrystals or crystalline complexes with the same active pharmaceutical ingredient (API) will have different solubilities and dissolution rates depending on the nature of the other reactive components in the molecular complex. The solubility and dissolution rate of a family of cocrystals with the same API and a series of ligands that are weak acids or weak bases can be controlled by the acid or base association constant of the ligand and the pH of the dissolution medium. In various embodiments, methods are provided of imparting pH dependent dissolution to non-ionizable APIs or to modulate the dissolution of ionizable APIs.

In one embodiment, the current teachings provide methods of decreasing the solubility of a molecular complex in a solvent system. The molecular complex is made of two or more organic reactants held in complex by nonionic interactions between the reactants. In one aspect the method involves adding a stoichiometric excess of one of the reactants to a solution of the complex, or to a solution of the components in solution in a stoichiometric amount equal to the presence of the components in the cocrystal, optionally with adjustment of the pH in a system where at least one of the cocrystal components is ionizable as a weak acid or a weak base. In various embodiments, the complex is formed by conventional methods or by methods described herein. In various embodiments, the method involves adding a composition including a solid reactant to a solution of the other reactant(s) or a solution of the complex. The solid reactant can alternatively be added as a solid or as the solid phase of a suspension or slurry. In various embodiments, it is possible but not required that the multi-component complex forms in solution before it precipitates as the solid cocrystal.

If used, the stoichiometric excess is preferably at least 2:1, more preferably 5:1, and more preferably 10:1, based on the stoichiometric presence of the reactant in the complex. For example, in the common situation where the complex is a binary (1:1) complex, it is preferred to add at least 2 moles of reactant per mole of complex, preferably at least 5, and more preferably at least 10 to achieve the respective ratios. To illustrate for a ternary (e.g. 2:1) complex, a 2:1 stoichiometric excess is achieved by providing 4 moles of the first reactant (the one present twice in the complex) to one mole of the other reactant.

Somewhat more generally, a method for precipitating a molecular complex from a solvent system, the complex comprising two or more reactants held in complex by nonionic interactions, involves adding a stoichiometric excess such as described above of one of the reactants to a solution comprising the two or more reactants, optionally adjusting the pH to a lower level to enhance precipitation of a molecular complex containing a weak acid or to a higher level if the complex contains a weak base. In a preferred embodiment, the solution comprising the two or more reactants contains the respective reactants at stoichiometric amounts corresponding to their presence in the complex. Preferably, the solution is relatively concentrated in at least one of the reactants. For example, at least one of the reactants is saturated.

In another aspect cocrystal methods involve combining the reactants and a liquid solvent under over saturation conditions with respect to the molecular complex in the solvent. One of the reactants is present in the combination in at least a 2:1 molar excess relative to its presence in the cocrystal, preferably 5:1, and more preferably 10:1 molar excess. Preferably, the reactants and liquid solvent are combined under over saturation conditions. Here and elsewhere, over saturation conditions means that the concentrations of the individual reactants in the reactant/solvent system are such that when a molecular complex forms, it is formed at a concentration at or above its solubility in the solvent. As noted herein, it is believed that providing one of the reactants (not both or all) in a molar excess, preferably a significant molar excess, leads to lower solubility of the molecular complex in the solvent, and an increased likelihood that over saturation conditions are reached with respect to the complex. Accordingly, it is preferred to provide at least one of the reactants and preferably both in relatively concentrated forms. Optionally, pH is maintained or adjusted as appropriate to promote or enhance precipitation or dissolution as desired.

Here and in other embodiments, when the solubilities of the reactants in the solvent used differ, highly concentrated or even saturated solutions of both of the reactants can be used. This provides not only a high concentration to achieve over saturation conditions, but also a high molar excess of one of the components, which is believed to lower the solubility of the complex in the solvent and lead to its precipitation as a cocrystal.

In another embodiment, the teachings provide for making pharmaceutical cocrystals by precipitation of a solid form from a solvent. The pharmaceutical cocrystal is a solid molecular complex between two or more reactants held together by nonionic interaction, wherein one of the reactants is an active pharmaceutical ingredient. The method involves combining the active pharmaceutical ingredient, other reactants and solvent under over saturation or supersaturation conditions with respect to the complex in the solution. As before, at least one of the active pharmaceutical ingredients and the other reactant or reactants is provided in a molar excess of at least 2:1 with respect to its presence in the complex, preferably at least 5:1, and more preferably at least 10:1. With respect to identity of the individual reactants, the component provided in molar excess in various embodiments is either the active pharmaceutical ingredient or another reactant. Optionally, pH is maintained or adjusted as desired to enhance or inhibit dissolution or precipitation of API cocrystals or molecular complexes containing an ionizable component.

In another embodiment, the reaction cocrystallization methods are used to screen for the formation of a nonionic molecular complex from prospective reactants. In various embodiments, the methods are used to identify conditions under which known or prospective complexes precipitate (or do not precipitate) from various solvents. Alternatively or in addition, the screening methods are used to discover or synthesize new complexes or cocrystals. Thus, pairs, trios, or other combinations of prospective reactants are tested in the method. Prospective reactants are combined in a solvent, wherein the prospective reactants are present at approximately stoichiometric levels or one of the prospective reactants is preferably present in solution at a molar excess preferably at a ratio of at least 2:1 with respect to another reactant, based on the molar or stoichiometric presence of the reactant in the cocrystal. If one of the components is ionizable as a weak acid, the pH is optionally maintained at or adjusted to a pH that is at or below the pKₐ of the weak acid component. If one of the components is ionizable as a weak base, the pH is similarly adjusted to or maintained at a value of about the pKa or preferably at least one or at least units above the pKₐ of a weak base component. The pH is in this way adjusted to favor precipitation of a cocrystal containing an ionizable component.

Upon combining the prospective reactants at the desired pH, the system is observed. If a precipitate forms, the precipitate is analyzed to determine or confirm that a cocrystal form comprising the reactants has come out of solution. In various embodiments, the precipitate is analyzed by Raman spectroscopy, infrared spectroscopy, x-ray diffraction, or other suitable procedures. In some embodiments, a cocrystal phase (precipitated molecular complex) exhibits a different Raman absorption spectrum than either of the reactants. In a non-limiting example, the presence of a reactant in a cocrystalline form is detected by observing shifts in Raman peaks or infrared absorption bands that can be on the order of 1 to 10 wave numbers. In the case of x-ray diffraction, it is well known that cocrystals crystallize in different unit cells than the reactants from which the complexes are formed. The different unit cell dimensions can be determined by x-ray diffraction methods such as powder diffraction.

Here and in other embodiments, when the individual prospective reactants have different solubilities in the solvent or solvent system to be used, the reactants are preferably provided at relatively concentrated levels, up to and including saturation in the solvent. Where the solubilities differ widely, saturated solutions of the individual reactants can provide suitable molar ratios of at least 2:1, at least 5:1, or at least 10:1 of the prospective reactants in the solvent (all ratios are in relation to the molar presence of the respective reactants in the molecular complex). As noted, the use of relatively concentrated solutions of the individual reactants increases the likelihood that the molecular complex formed in solution is at or above its solubility limit, or that over saturation conditions are achieved. Also as noted, the use of prospective reactants where one is in a molar excess lowers the solubility of the complex and increases the likelihood that the complex is the least soluble form in the solution, leading to its precipitation. In addition, adjusting or maintaining the pH as described changes the solubility of molecular complexes containing ionizable components.

In various embodiments, the methods of the invention involve combining reactants in a solvent system in such a way that the stoichiometric presence of one of the reactants in solution is greater than the other reactant or reactants, when measured relative to the stoichiometric presence of the reactant component in the complex to be formed. This imbalance of stoichiometry in solution is believed to lead to precipitation of molecular complexes (cocrystals), as discussed theoretically below. The reactants are combined in various forms: as solutions, as slurries, or as solids. In various embodiments, a liquid or a vapor is brought into contact with solid ingredients. Differential solubility of the reactants in the liquid solvent (or the vapor as is adsorbs on to the solid) leads to non-stoichiometric concentrations of reactants in solution, which lead to enhanced precipitation and isolation of cocrystal.

In various embodiments, combining the reactants with solvent is accomplished by sorption. Sorption is the spontaneous acquisition of a component (water, ethanol or another solvent) from the atmosphere or vapor phase. Sorption of a component from the vapor can take the form of a condensed phase and can serve as a solvent. For instance, in the case of water, many water soluble substances have been shown to spontaneously dissolve when exposed to relative humidity above a critical value, where adsorbed water serves as a solvent. The sorbed phase provides a microphase for reaction cocrystallization as a result of dissolution of reactants, non-stoichiometric concentrations of reactants in solution and precipitation of the molecular complex in crystal form. The reaction mediated by vapor sorption onto a solid (solvents such as ethanol, water, and others) or by a deliquescent behavior will proceed to completion, or consume the reactants if the reactants in solid state are in the same stoichiometry as in the molecular complex to be crystallized. For crystallization of the molecular complex, non-stoichiometric solutions (i.e. those in which there is a molar excess of one of the reactants in solution) are achieved by different dissolution rates or solubilities of the reactants in the sorbed phase or solvent film.

In preferred embodiments, the cocrystalline product of the invention contains pharmaceutical components or active pharmaceutical ingredients. Advantageously, the reaction cocrystallization proceeds with various solid state forms of the reactants, such as polymorphs, salts, hydrates, solvates, amorphous, or crystalline solid state forms.

In various embodiments, the solid state forms of the reactants are used in the solid state, in slurries in contact with a liquid phase, or in solution. It is to be understood that stoichiometric, excess refers to the solution concentration of reactants. Therefore reactants, when all reactants are in solid forms, can have the stoichiometric composition in solid phase equal to that of the cocrystal; in such a case, however, non-stoichiometric solution concentrations are achieved by different dissolution rates of each reactant in the solvent.

In various embodiments, the invention provides batch and continuous cocrystallization reactions by, for example, slurrying one or more of the reactants in solvent or solutions of reactant(s) to a suspension (slurry) of reactant(s), and for adding pure solvent to solid reactant, including contacting solvent with solid reactants or adding solvent in a larger phase to prepare a slurry of the reactants.

In various embodiments, methods of the invention are carried out by combining two or more reactants with a solvent to form a reaction cocrystallization system, with optional pH adjustment on the system or on the various streams.

In various embodiments, reaction cocrystallization is carried out by combining various streams into suitable reactors, vessels, mixers, and the like. The various streams together comprise the reactants (cocrystalline components) and solvent. As desired, the streams are adjusted to neutral, acid, or basic pH. Normally, individual streams before combination comprise predominantly one or another reactant. For example (illustrating with a complex containing two components A and B), a first stream comprising cocrystal component A is provided, and a second stream comprising cocrystal component B is provided. For cocrystals containing more than two components a third stream containing cocrystal components C is provided. In preferred embodiments, the respective streams before combination contain only the respective cocrystal component; alternatively, the individual streams contain other cocrystal components (reactants). While the method is general for ternary and higher complexes, the method is further described herein for illustrative purposes with reference to a binary system containing two reactants A and B.

Streams comprising reactants A or B are provided as a pure solid, as a solution of the respective reactant in a solvent, or as a suspension or slurry. When provided as a suspension, the streams contain solid reactants in contact with a solvent phase, the solvent phase normally containing dissolved reactant. For example, a slurry stream contains a reactant in contact with a saturated solution of the reactant in the solvent. When the streams collectively containing reactants A and B are combined, a reaction cocrystallization mixture is produced that contains reactant A, reactant B, and a solvent. The pH of the combined streams is controlled by the pH of the individual streams, and by any acid or base added.

Any manner of combining the streams can be used, as long as a reaction cocrystallization mixture containing the reactions and solvents is produced. In various embodiments, the cocrystallization mixture is a solution of reactants in which the presence of one of the reactants is in a molar excess relative to its presence in the complex formed from nonionic interactions between the reactants. As noted, the molar excess is preferably 2:1 or greater in solution. For example, in some embodiments, solutions of reactants, which in various embodiments are premixed prior to combination, are fed into a reactor or vessel. In various embodiments, the solutions are saturated, supersaturated, or undersaturated at the temperature of feeding. If desired, the solutions are prepared warm and allowed to cool prior to or after addition to the vessel. In this way, over saturation or supersaturation conditions are readily achieved. In a batch process, the reaction cocrystallization mixture is formed in a reactor or vessel and a precipitate containing the cocrystalline composition is isolated by draining or filtering.

In various embodiments, continuous processes are carried out, for example in a tubular reactor or vessel. Streams collectively containing reactants A and B are continuously pumped into a reactor where pH is optionally adjusted, reaction takes place, and cocrystals are precipitated. The precipitate containing the cocrystalline composition is continuously renewed by filtration or other means.

Screening methods take advantage of the increased efficiency of precipitation and higher yields brought about by combining cocrystal component non-stoichiometrically and/or at low or high pH as described herein. In one aspect, screening involves subjecting a complex or a cocrystal system to a series of conditions to determine whether and under what conditions a cocrystal precipitate is formed and isolated. Conditions to be evaluated include, without limitation, nature of the co-reactant, solvent, yield of cocrystal precipitate, molar ratio of the reactants, pH and so on. The methods are adaptable to high throughput operations and/or robotic automation. In one embodiment, the screening methods are carried out in conventional equipment, such as an industry standard 96-well plastic tray.

In various embodiments, screening methods involve variation of cocrystallization conditions according to a predetermined or pre-set plan for probing the response of the cocrystallization system to experimental variables. In a non-limiting embodiment, the plan is to select an active pharmaceutical agent and probe what organic molecules form an isolatable cocrystal upon precipitation from a solvent system. Here a series of reaction vessels (which can be the individual wells of a multi-well plate) are provided with a solution, slurry, or solid comprising the active pharmaceutical agent of interest. Then, a series of test solutions, test slurries, or test solids is combined with the pharmaceutical agents in the respective reaction vessels.

In one embodiment, the experimental conditions are selected such that, upon combination of the prospective reactants, one of the reactants is in a molar excess, preferably of at 2:1, and more preferably at least 5:1, with respect to its molar presence in the complex. The use of a stoichiometric excess of one of the reactants increases the likelihood that any nonionic complex formed will be the least soluble form in the system, and so will precipitate out as a cocrystal.

The nature of any precipitate is then probed by any of a number of suitable analytic techniques, such as without limitation Raman spectroscopy, infrared spectroscopy, FTIR spectroscopy, and x-ray diffraction. In various embodiments, the analysis involves determination of the structure and composition of the precipitate (e.g. to demonstrate that the precipitate is a cocrystalline form and not just one or other of the reactants). When the cocrystal or cocrystal system under investigation is known, the analysis can be limited to a confirmation that the cocrystal precipitate at hand is the same as that noted before. For example, analysis can be limited to a region of the spectrum of diffraction pattern known to contain diagnostic peaks, such as a particular Raman or infrared band, or a particular set of diffraction peaks indicative of the structure. In some embodiments, the precipitate is analyzed at a single wave length or diffraction angle to determine, at least on a first pass, whether the precipitate is of interest or whether the particular conditions are worthy of further study.

The methods also lend themselves to use in combinatorial screening. For simplicity, the method is described for a binary (two-component) cocrystal system, but the results are readily generalized to three-component and higher systems. Instead of a single component A being combined with a single component B, and any resulting precipitate being analyzed to confirm or determine whether the A:B is formed, the streams containing A can instead contain a plurality of components A₁, A₂, A₃ . . . Aᵢ. Similarly, the streams containing B optionally contain a plurality of components B₁, B₂, B₃ . . . Bⱼ. In some embodiments, a plurality of components Aᵢ is screened with a single component B, and vice versa. For example, component A comprises a drug or drugs of interest, and component B comprises a potential complex former or collection of potential complex formers with the active drug(s) A. In some embodiments, a plurality of components Aᵢ is screened with a plurality of components Bⱼ.

The compositions of the respective combinatorial libraries of potential reactants, the solubility of individual components in the solvents chosen, the nature of the complexes formed (e.g. whether binary, ternary, or higher), and other factors determine the conditions under which the screening is to take place. The key is to provide the potential reactants in a stoichiometrically unbalanced way (i.e. with one of the complex formers or potential complex formers in stoichiometric excess, preferably 2:1 or greater, relative to its presence in the cocrystal), and/or under pH conditions, that affect cocrystal solubility in order to increase the likelihood that a complex formed will have lower solubility than the reactants, and thus preferentially precipitate.

When the screening is carried out combinatorially and a positive result is achieved (i.e. when a precipitate is observed) in a particular reaction vessel, generally the system needs to be studied further to determine which of the reactants Aᵢ and Bⱼ (and Cₖ when ternary systems are investigated) were responsible for the precipitate formation. As appropriate, further combinatorial or one-on-one screening is carried out in a further investigation.

In various embodiments, combinatorial screenings are carried out to determine conditions under which a complex formation is desirably avoided. To illustrate, it is sometimes useful to determine whether a particular active ingredient is capable of forming a new solid form in combination with any of the ingredients the active comes into contact with during synthesis, compounding, or administration. To probe this, a single reactant A (for example, the active ingredient of interest) is combinatorially screened with a number of components Bⱼ.

Cocrystals containing an active pharmaceutical ingredient (API) as one component or reactant are known and include the cocrystals listed in the table. Many of the cocrystals contain a weak acid or a weak base as a component. The “co-reactant” along with the API is referred to as a “ligand” in the table. The table also indicates the composition of the complex as a ratio of API:ligand. This ratio gives the stoichiometric presence of the individual reactants in the complex.

| | | Ratio |
|---|---|---|
| API | Ligand | (API:Ligand) |
| Carbamazepine | Nicotinamide | 1:1 |
| | Saccharin | 1:1 |
| | Benzoquinone | 2:1 |
| | Terephthalaldehyde | 2:1 |
| | Acetic acid | 1:1 |
| | Formic acid | 1:1 |
| | Butyric acid | 1:1 |
| | Trimesic acid | 1:1 |
| | 5-nitroisophthalic acid | 1:1 |
| | Adamantane-1,3,5,7- | 2:1 |
| | tetracarboxylic acid | |
| | Formamide | 1:1 |
| Caffeine | Malonic acid | 2:1 |
| | Oxalic acid | 2:1 |
| | Glutaric acid | 1:1 |
| | Maleic acid | 2:1, 1:1 |
| | Benzoic acid | 1:1 |
| | Salicylic acid | 1:1 |
| | p-hydroxybenzoic acid | 1:1 |
| | m-hydroxybenzoic acid | 1:1 |
| | Gentisic acid | 1:1, 1:2 |
| Itraconazole | Succinic acid | 2:1 |
| | Malic acid | 2:1 |
| | Tartaric acid | 2:1 |
| | Fumaric acid | 2:1 |
| Fluoxetine hydrochloride | Succinic acid | 2:1 |
| | Fumaric acid | 2:1 |
| | Benzoic acid | 1:1 |
| Aspirin | 4,4′-bipyridine | 2:1 |
| Ibuprofen | 4,4′-bipyridine | 2:1 |
| Flurbiprofen | 4,4′-bipyridine | 2:1 |
| | trans-1,2-bis(4-pyridyl)ethylene | 2:1 |
| | 4,4′-dipyridylethane | 2:1 |
| Sulfamethazine | Benzoic acid | 1:1 |
| | Salicylic acid | 1:1 |
| | Anthranilic acid | 1:1 |
| | Acetylsalicylic acid | 1:1 |
| | o-phthalic acid | 1:1 |
| | p-chlorobenzoic acid | 1:1 |
| | p-aminobenzoic acid | 1:1 |
| | p-aminosalicylic acid | 1:1 |
| | Aminacrine | 1:1 |
| | Trimethoprim (methanolate) | 1:1 |
| | Trimethoprim (monohydrate) | 2:1 |
| Sulfamethoxypyridazine | Trimethoprim | 1:1 |
| Sulfametrole | Tetroxoprim (hydrate, | 1:1 |
| | ethanolate, methanolate) | |
| | Trimethoprim | 1:1 |
| Sulfamethoxazole | Trimethoprim | 1:1 |
| Theophylline | Ethylenediamine | 2:1 |
| | 1,10-phenanthroline | 1:1 |
| | ethylenediamine carbamate | 1:1 |
| | Salicylic acid | 1:1 |
| | 5-sulfosalicylic acid (hydrate) | 1:1 |
| | p-nitroaniline | 1:1 |
| | Urea | 1:1 |
| | Sulfathiazole | 1:1 |
| | 5-chlorosalicylic acid | 1:1 |
| Mebandazole | Propionic acid | 1:1 |

The case of fluoxetine hydrochloride illustrates a case where one of the reactants that make up a molecular complex is itself a kind of multi-component system, here a salt. The complex is held together by nonionic interactions between the respective ligands and the salt. It is also noted that several of the ligands in the table above are carboxylic acids. The complexes are formed under conditions of solvent and pH where a salt does not form upon complexation. For example, salts of carboxylic acids and weak amine bases generally do not form in non-aqueous solvents. In aqueous solutions, the pH determines the ionization state of weak acids and weak bases, according to known principles. In the table, the carboxylic acid ligands illustrate suitable functional groups that can form nonionic interactions by hydrogen bonding, dipole-dipole interactions, and the like.

According to various aspects of the current teachings, dissolution of cocrystals and its rate are influenced by and depend on the pH of a solvent system in contact with the cocrystal. Although a cocrystal involves different neutral molecules, if one or both components of the cocrystal is ionizable; the solubility of the cocrystal will be dependent on the solution pH. For the sake of simplicity, the concept will be illustrated by description of the pH-solubility profile of a cocrystal where one of the components is a monoprotic acid.

Consider a cocrystal HA:B where the components HA and B are in 1:1 ratio and HA is a monoprotic weak acid. B and HA represent the API and ligand, respectively. The equilibrium equations for cocrystal dissociation (assuming no complexation in solution) and ionization of weak acid HA are as given below:

$$\begin{matrix}HA \text{:} {B}_{solid} ⇌ {HA}_{soln} + {B}_{soln} \\ {K}_{sp} = [ HA ] [ B ] & ( 1 ) \\ {HA}_{soln} ⇌ {A}_{soln}^{-} + {H}_{soln}^{+} \\ {K}_{a} = \frac{[ {H}^{+} ] [ {A}^{-} ]}{[ HA ]} & ( 2 )\end{matrix}$$

where Kₛₚ is the solubility product of the cocrystal and Kₐ is the dissociation constant of the weak acid.

In the absence of complexation in solution, the total concentration of B in solution is equal to the concentration of free B in solution. Also, under stoichiometric conditions and in the absence of precipitation of single components, the total concentration of A and B in solution will be equal to the cocrystal solubility. Thus,

[A]_{T}=[B]_{T}=S_{cocrystal}.

Mass balance for the total concentration of drug B in solution is given by:

[B]_{T}=[A]_{T}=[HA]+[A] (3)

Equation (3) can be re-written by substituting for [HA] and [A⁻] from equations (1) and (2), respectively. Thus,

$${[ B ]}_{T} = {[ A ]}_{T} = [ HA ] ( 1 + \frac{{K}_{a}}{[ {H}^{+} ]} )$$

or by substituting the equilibrium solubility constant (Ksp)

$${[ B ]}_{T} = \frac{{K}_{sp}}{[ B ]} ( 1 + \frac{{K}_{a}}{[ {H}^{+} ]} ) \qquad ( 4 )$$

under the conditions stated above, [B]=[B]_{T} and [A]_{T}=[B]_{T}=S_{cocrystal}. Re-arranging equation (4) yields an expression for the cocrystal solubility dependence on [H⁺], Ka and Ksp.

$${S}_{cocrystal} = \sqrt{{K}_{sp} ( 1 + \frac{{K}_{a}}{[ {H}^{+} ]} )} \qquad ( 5 )$$

Since pH=−log [H⁺] the pH dependent solubility can be easily calculated.

Dissolution rate is proportional to the solubility and is given by

Dissolution rate=kAS

where k is a dissolution rate constant, A is the solid surface area and S is the cocrystal solubility.

Equation 5 predicts that the cocrystal solubility increases as [H+] decreases or as pH increases. Ksp is the cocrystal solubility product and Ka is the acid dissociation constant of the cocrystal former. Since cocrystal dissolution rate is directly proportional to solubility, similar behavior is predicted.

A similar analysis for a cocrystal with a neutral weak base predicts that solubility and dissolution rate increase as [H⁺] increases or as pH decreases. This solubility behavior is opposite to that of salts where both components are ionized in the crystalline state.

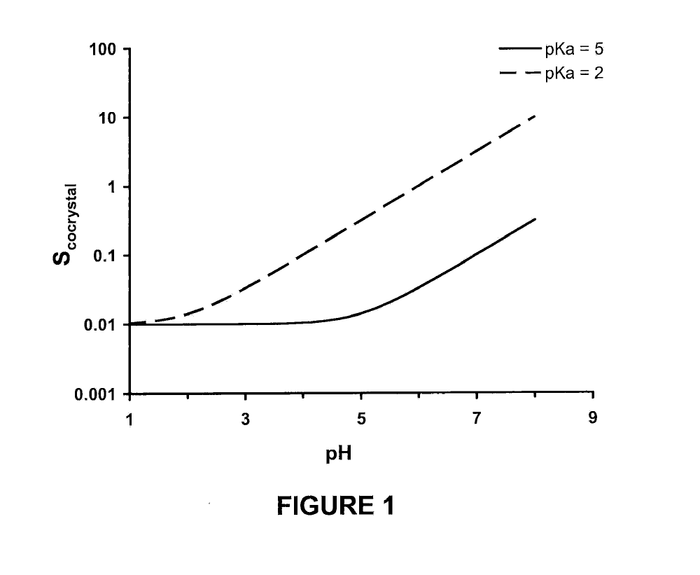

FIG. 1 shows the prediction of the above equation for the solubility or dissolution dependence on pH for a cocrystal with neutral molecules HA and B, where B is the non-ionizable API and HA is the weak acid. FIG. 1 shows pH-solubility dependence for a cocrystal of a non-ionizable API and weakly acidic ligands with pKa values of 2 and 5, given an illustrative cocrystal Kₛₚ=1×10⁻⁴ M².

This plot shows that by lowering the pKa of the cocrystal former (HA), higher cocrystal solubilities are achieved at lower pH values. At pH<<pKₐ, the cocrystal solubility is at its lowest intrinsic solubility value, given by (Kₛₚ)^{1/2}. At pH=pKₐ, the cocrystal solubility is 1.4 times higher, and at pH>>pKₐ, the solubility increases exponentially. Also, increasing the Kₛₚ value increases the intrinsic solubility of cocrystal. The Kₛₚ value is characteristic of the cocrystal of an API with a specific ligand. Therefore, if multiple cocrystals exist for the same API, determination and comparison of the Kₛₚ and Kₐ values enables one to select the cocrystal with the desired solubility and dissolution rate pH dependence.

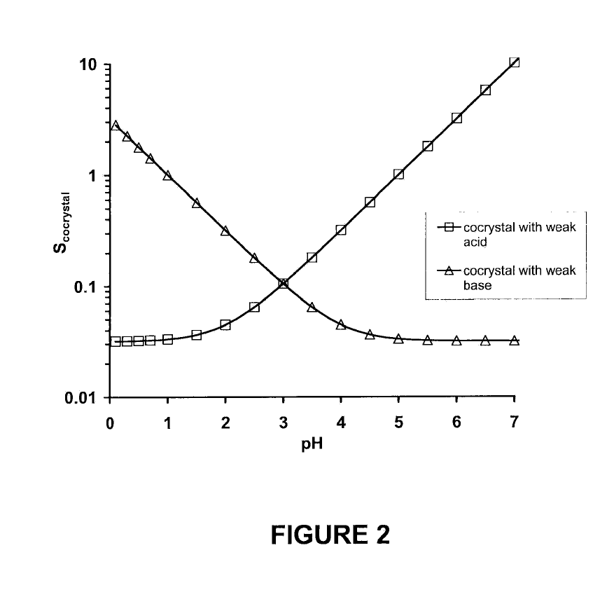

FIG. 2 shows the solubility behavior for a cocrystal with a non-ionizable API and a weakly basic ligand or cocrystal former and for a weakly acidic ligand. The acid dissociation constant for the conjugate acid of the weak base is given by Ka2. In this case, the solubility or dissolution of cocrystal is constant at pH>>pKa₂ and increases for pH<<pKa₂.

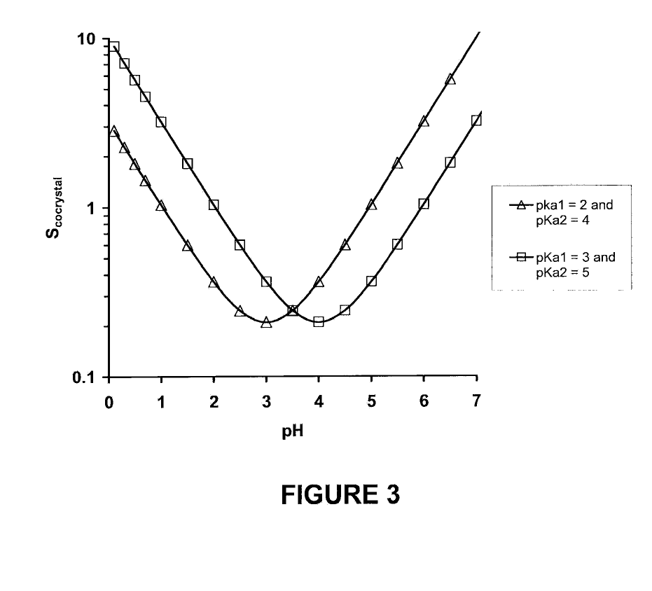

FIG. 3 shows the solubility pH dependence for a cocrystal with neutral components where both components are ionizable in solution; or where one of the components has functional groups that can ionize in solution; one is a weak acid and the other is a weak base. The cocrystal solubility and dissolution rate dependence on pH will have the U shape curve shown.

These models show that cocrystal solubility and dissolution rates can be tailored by the choice of cocrystal component pKa values and by considering the simultaneous equilibrium expressions between cocrystal dissociation and ionization of its components in solution as described below.

In various embodiments, pH adjustment is used in conjunction with excess stoichiometry to control dissolution and formation of cocrystals. Although the invention is not to be limited thereby, a theoretical explanation of the effect is given in U.S. Ser. No. 11/262,959 filed on Oct. 31, 2005, the full disclosure and figures of which are hereby incorporated by reference.

### EXAMPLES

### Example 1

### CBZ-SAC (Carbamazepine-Saccharin) Cocrystallization by Lowering pH

0.2 g (0.97 mmol) sodium saccharin is dissolved in 3 mL unbuffered water, resulting in a pH of 6.3. 0.16 g (0.69 mmol) carbamazepine anhydrous (form III) is added to the sodium saccharin solution. The pH is lowered by addition of HCl, to a pH of 1.1. This process is carried out at room temperature. Cocrystal formation is monitored on-line by Raman spectroscopy and confirmed by comparing spectra to a reference cocrystal spectrum. Cocrystal is formed within 10 minutes.

### Example 2

### CBZ-4ABA-HYD (Carbamazepine-4Aminobenzoic Acid Hydrate) Cocrystallization by Lowering pH

0.05 g (0.21 mmol) carbamazepine anhydrous (form III) is added to 3 mL of a buffered phosphate solution of pH 6.6. 0.04 g (0.29 mmol) of 4-aminobenzoic acid is dissolved and the solution pH is lowered to 3.2 by addition of HCl. This process is carried out at room temperature. The transformation to cocrystal is monitored by Raman spectroscopy and confirmed by comparing spectra to a reference cocrystal spectrum. Cocrystal is formed within 5 minutes.

### Example 3

### CBZ-4ABA-HYD Cocrystallization by Raising pH

0.05 g (0.21 mmol) carbamazepine anhydrous (form III) is added to 3 mL of a solution of pH 1.1. 0.04 g (0.29 mmol) 4-aminobenzoic acid is added to the solution. NaOH is added to raise the pH to 3.6. This process is carried out at room temperature. The transformation to cocrystal is monitored by Raman spectroscopy and confirmed by comparing spectra to a reference cocrystal spectrum. Cocrystal is formed within 5 minutes.

Use of higher ligand concentration (higher ratio compared to API) will lead to higher cocrystal yields and faster rates of cocrystal formation. Higher yields and faster rates of formation are also achieved by increasing the API concentration. If the solubility of either component is limiting, temperature can be increased to allow dissolution of components and the change in pH carried out under isothermal conditions.

What is claimed is:

1. A method for manufacturing a cocrystal, wherein the cocrystal comprises two or more component organic reactants held in complex by nonionic interactions, wherein at least one of the reactants is a weak acid, the method comprising
adding the component reactants of the cocrystal to a solvent system at an initial pH, and
simultaneously or subsequently then adjusting the pH to or maintaining the pH at a value at which the solubility of the crystalline complex formed between or among the reactants is lowered sufficiently that the crystalline molecular complex precipitates from the solvent as the cocrystal.

2. A method according to claim 1, comprising adjusting the pH of the solvent system to or maintaining the pH at a value of less than or equal to the pKa of the weak acid component.

3. A method according to claim 1, comprising adjusting the pH to or maintaining the pH at a value that is one unit or more below the pKa.

4. A method according to claim 1, comprising adjusting the pH to or maintaining the pH at a value that is two units or more below the pKa.

5. A method according to claim 1, wherein the molecular complex precipitates at a pH below or at the pKa plus two units.

6. A method according to claim 1, wherein the weak acid comprises a carboxylic acid group.

7. A method according to claim 5, wherein adding the component reactants comprises combining a plurality of streams that collectively provide at least a 5:1 molar excess of one of the reactants, relative to the amount of the reactant in the molecular complex.

8. A method according to claim 5, wherein one of the reactants is an active pharmaceutical ingredient.

9. A method for controlling the solubility of a crystalline complex in an solvent system, wherein the crystalline complex comprises two or more component organic reactants held in complex by nonionic interactions between the reactants, wherein at least one of the reactants is a weak base, the method comprising
adding the component reactants of the cocrystal to a solvent system at an initial pH, and
simultaneously or subsequently adjusting the pH to or maintaining the pH at a value at which the solubility of the crystalline complex formed between or among the reactants is lowered sufficiently that the crystalline molecular complex precipitates from the solvent as the cocrystal.

10. A method according to claim 9, comprising adjusting the pH of the solvent system to or maintaining the pH at a value of greater than or equal to the pKa of the weak base component.

11. A method according to claim 9, comprising adjusting the pH to or maintaining the pH at a value of one unit or more above the pKa.

12. A method according to claim 11, comprising adjusting the pH to or maintaining the pH at a value that is two units or more above the pKa.

13. A method according to claim 9, wherein the molecular complex precipitates at a pH at or above the pKa minus two units of the weak base.

14. A method according to claim 9, wherein the weak base comprises an organic amine group.

15. A method according to claim 9, comprising forming the molecular complex by combining a plurality of streams that collectively provide at least a 5:1 molar excess of one of the reactants, relative to the amount of the reactant in the molecular

16. A method according to claim 9, wherein one of the reactants is an active pharmaceutical ingredient.

17. A method of screening for the formation of a non-ionic molecular complex from prospective reactants, wherein at least one of the reactants is ionizable, comprising
combining the prospective reactants in a solvent, and simultaneously or subsequently
adjusting the pH to or maintaining the pH at a value below the pKa plus two units of the ionizable reactant if the reactant is a weak acid, or to or at a value above the pKa minus two units of the ionizable reactant if the reactant is a weak base; and
if a precipitate forms, analyzing the precipitate to confirm or determine that it is a cocrystal form comprising the reactants.

18. A method according to claim 17, comprising measuring a Raman band of the precipitate.

19. A method according to claim 17, comprising analyzing the precipitate by x-ray diffraction.

20. A method according to claim 17, wherein combining the prospective reactants comprises adding together separate solutions of the reactants.

21. A method according to claim 20, wherein the reactants have different solubilities in the solvent and the solution of the reactant provided at the lower molar ratio is saturated.

22. A method according to claim 17, wherein combining the perspective reactants comprises adding one reactant as a solid to a solution of another reactant.

23. A method according to claim 17, carried out combinatorially.

24. A method for controlling the dissolution of a crystalline complex in contact with a solvent system, wherein the crystalline complex comprises two or more components held in complex by nonionic interactions between the components, wherein at least one of the components is an ionizable weak acid or weak base, the method comprising
disposing the crystalline complex in contact with the solvent system, and simultaneously or subsequently thereto
adjusting the pH of the solvent system to or maintaining the pH at a value of equal to or greater than the pKa of the weak acid component, or a value of equal to or less than the pKa of the weak base component.
